(19) United States
(12) Patent Application Publication
(10) Pub. No.: US 2020/0152305 A1
(43) Pub. Date: May 14, 2020

(54) HEALTHCARE COMPLIANCE PROCESS OVER A NETWORK

(71) Applicant: VP Health Group, Inc., Naples, FL (US)

(72) Inventors: Brice Pelliccioni, Estero, FL (US); Paul Vance, Naples, FL (US)

(21) Appl. No.: 16/682,972

(22) Filed: Nov. 13, 2019

Related U.S. Application Data
(60) Provisional application No. 62/760,710, filed on Nov. 13, 2018.

(51) Int. Cl. G16H 20/10; G16H 40/67

(52) U.S. Cl. CPC G16H 20/10; G16H 40/67

(57) ABSTRACT

A method and system for increasing compliance with a prescribed treatment protocol is provided. The system includes a network operating over one or more servers configured to provide a portal that supports interactions with healthcare practitioners and their patients. The network is configured to receive a first plurality of real-time data associated with one or more patients in order to generate a prescribed treatment protocol. The network is further configured to provide a second plurality of real-time data associated with medication, supplements, and other applicable information relating to the prescribed treatment protocol. The network performs one or more analysis functions based on the pluralities of data in order to provide analytic and micro-analytic data relating to compliance with the prescribed treatment protocol to the healthcare practitioner and/or the one or more patients.

### CROSS-REFERENCE TO RELATED APPLICATIONS

The present U.S. Utility Patent Application claims priority pursuant to 35 U.S.C. § 119(e) to U.S. Provisional Application No. 62/760,710 entitled “HEALTHCARE COMPLIANCE PROCESS OVER A NETWORK”, filed 11-13-2018, which is hereby incorporated herein by reference in its entirety and made part of the present U.S. Utility Patent Application for all purposes.

### TECHNICAL FIELD

The technical field relates generally to healthcare and, more specifically, to automated processes for facilitating compliance with healthcare directives, and reporting of said compliance.

### BACKGROUND

In the United States alone, upwards almost 100 million Americans suffer from one or more medical conditions that require medication. Treatment of these medical conditions represents the majority of health care expenditures and the total direct costs of treating these medical conditions is estimated to rise to almost a trillion dollars by the year 2030.

Many patients with medical conditions requiring medication usually take their medication at home, school or work. Unfortunately, many patients may be unable to reliably manage their treatment, such as medication regimens, without the supervision of a healthcare provider. To be effective, medications often must follow various scheduling and dietary guidelines. For example, some medications are to be taken with food, others are not. Some medications are to be taken only once a day, others multiple times per day. Remembering when to take a medication and how much of it can become difficult as the number of concurrent medications increases.

On the healthcare provider side, it is crucial for physicians and healthcare professional to know exactly how much a patient has complied with a prescribed medical treatment. The extent of a patient's compliance can alter the way a physician treats a patient or prescribes additional regimens. Patient compliance also affects how long a medical condition persists and how it is monitored.

Unfortunately, existing methods and devices for assisting patients in following medication regimens, and for reporting patient compliance to healthcare providers, can be somewhat expensive and complex in design. Furthermore, existing methods and devices for assisting patients in following medication regimens, and for assisting healthcare professionals in monitoring patient compliance, can seem somewhat intrusive to a user.

Therefore, a need exists for improvements over the prior art, and more particularly for improved methods and systems for verifying a patient's compliance with a prescribed treatment protocol, and reporting said compliance, over a network.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### SUMMARY

A method and system for increasing compliance with a prescribed treatment protocol is provided. The system includes a network operating over one or more servers configured to provide a portal that supports interactions with healthcare practitioners and their patients. The network is configured to receive a first plurality of real-time data associated with one or more patients in order to generate a prescribed treatment protocol. The network is further configured to provide a second plurality of real-time data associated with medication, supplements, and other applicable information relating to the prescribed treatment protocol. The network performs one or more analysis functions based on the plurality of data in order to provide analytic and micro-analytic data relating to compliance with the prescribed treatment protocol to the healthcare practitioner and/or the one or more patients.

In one embodiment, the system for use by healthcare practitioners and patients for increasing compliance with a prescribed treatment protocol comprises: a) an application executed by a healthcare practitioner, the application configured for: 1. providing a dashboard that displays real-time data regarding patient compliance with a prescribed treatment protocol by individual and by group; 2. providing a dashboard that displays real-time data regarding sales of medication and supplements to patients with a prescribed treatment protocol by individual and by group; 3. providing a dashboard that displays real-time data regarding patient-derived revenue by individual and by group; 4. providing a dashboard that allows the healthcare practitioner to sort and view patients and related statistics using any of the data above; and 5. providing a communication functionality that allows the healthcare practitioner to communicate directly with their patients; b) a mobile application executed by a patient, the mobile application configured for: 1. providing an interface to allow the patient to enter his compliance with a prescribed treatment protocol; 2. providing an interface to allow the patient to communicate directly with their healthcare practitioner; 3. providing an interface to allow the patient to link or sync their mobile application with other health devices/trackers; and 4. providing an interface to allow the patient to purchase medication and supplements associated with their prescribed treatment protocol; c) a central server connected to the application and the mobile application, the central server configured for: 1. storing a user profile for each healthcare practitioner and each patient; 2. storing and serving any data related to any dashboard above; and 3. providing a communications platform for allowing any healthcare practitioner to communicate directly with patients.

In one embodiment, the system for use by healthcare practitioners and patients for increasing compliance with a prescribed treatment protocol includes one or more servers communicatively coupled to one or more computing devices configured to function as a centralized service for the network. The computing devices may be associated with healthcare practitioners and patients allowing a healthcare practitioner to not only generate and update the prescribed treatment protocol for patients, but also generate one or more analyses based on the first and/or second pluralities of real time data resulting in efficient and up to date communications exchanged between the healthcare practitioners and patients relating to compliance with the prescribed treatment protocol.

The system and method described herein provide improvements to the production, transmission, and presentation of data relating to prescribed treatment protocols in real time. The system and method support scalability of reports and analytics provided by the network via the faster processing and efficient centralization of analytical data associated with prescribed treatment protocols. The system also reduces human error associated with either healthcare practitioner entry and updating of the data or patient's failure to provide data relating to compliance with their prescribed treatment protocol. Thus, the system and method described herein improve the functioning of computing systems, and in particular computer systems in the medical industry, by enhancing the speed, accuracy, and efficiency of processing and presenting data analytics in real time.

### BRIEF DESCRIPTION OF THE DRAWINGS

The accompanying drawings, which are incorporated in and constitute a part of this disclosure, illustrate various example embodiments. In the drawings:

FIG. 1 is a diagram of an operating environment that supports a method and system for increasing compliance with a prescribed treatment protocol, according to an example embodiment;

FIG. 2 is a block diagram showing the data flow of the process for increasing compliance with the prescribed treatment protocol, according to one embodiment.

FIGS. 3A-3H are graphical user interfaces used the method and system for increasing compliance with a prescribed treatment protocol, according to an example embodiment;

FIG. 4 is a flow chart depicting a process for increasing compliance with a prescribed treatment protocol, according to an example embodiment.

FIG. 5 is a block diagram depicting a system including an example computing device and other computing devices.

Like reference numerals refer to like parts throughout the several views of the drawings.

### DETAILED DESCRIPTION

The following detailed description refers to the accompanying drawings. Wherever possible, the same reference numbers are used in the drawings and the following description to refer to the same or similar elements. While embodiments may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The claimed subject matter improves upon the problems with the prior art by providing a system and method for allowing a physician or other healthcare professional to confirm that a patient has been complying with a prescribed treatment protocol in between office visits. When a patient returns to see a physician or other healthcare professional for a follow up visit, the claimed subject matter provides highly accurate data regarding the current status of the patient, his or compliance with the prescribed regimen and the efficacy of the regimen in treating the patient's affliction. Further, the claimed subject matter provides a precise and automated way for a healthcare professional to view other key data, such as sales of medication, patient-derived revenue, and patient statistics. Also, the claimed subject matter allows for direct communication between healthcare professionals and patients. Thus, the claimed subject matter allows a doctor providing care to have a full and accurate picture of the efficacy of the prescribed regimens on his patients.

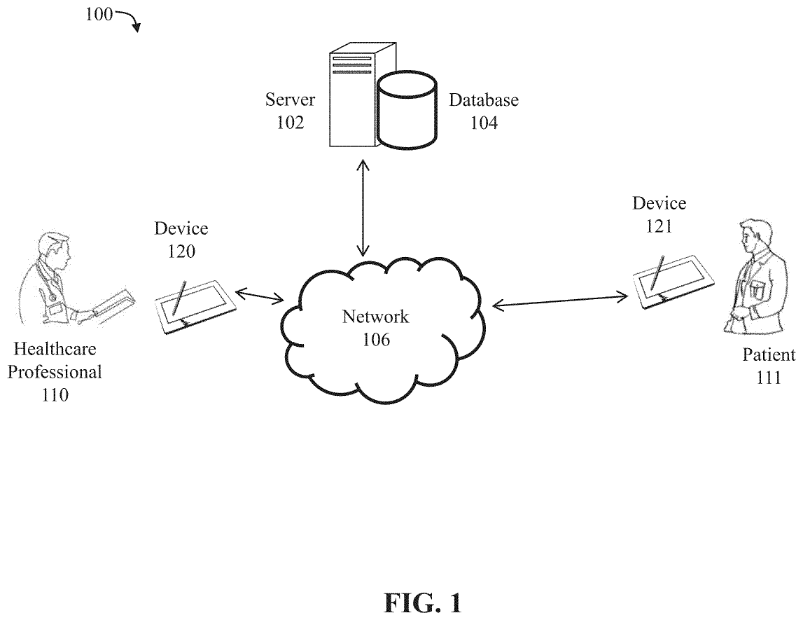

The illustration of FIG. 1 depicts an example system for increasing compliance with a prescribed treatment protocol 100 in which the techniques described may be practiced according to certain embodiments. In one embodiment, the system 100 may be implemented in hardware, software, or a combination of hardware and software. In some embodiment, the various components of the system 100 are implemented at least partially by hardware at one or more computing devices, such as one or more hardware processors executing instructions stored in one or more memories for performing various functions described herein. For example, descriptions of various components (or modules) as described in this application may be interpreted by one of skill in the art as providing pseudocode, an informal high-level description of one or more computer structures. The descriptions of the components may be converted into software code, including code executable by an electronic processor. The system 100 illustrates only one of many possible arrangements of components configured to perform the functionality described herein. Other arrangements may include fewer or different components, and the division of work between the components may vary depending on the arrangement.

As mentioned heretofore, FIG. 1 is a diagram of an operating environment that supports a system and method for increasing compliance with a prescribed treatment protocol, in an embodiment. The system 100 includes computing device 120 configured to be associated with a healthcare professional 110 and computing device 121 configured to be associated with a patient 111. Computing devices 120 and 121 are communicatively coupled either wirelessly or in a wired or fiber optic form via a network 106. In one embodiment, the network 106 may be implemented by a network of communicatively coupled computing devices associated with a server 102. Server 102 may include a database or repository 104, which may be a relational database comprising a Structured Query Language (SQL) database stored in a SQL server or any other applicable data structure, and may be distributed over one or more nodes or locations that are connected via network 106. Network 106 may utilize known security precautions such as encryption, passwords, limited Wi-Fi range, and the like. In one embodiment, network 106 may be configured to access computers and databases outside of system 100.

Although only server 102, database 104, and computing devices 120 and 121 are depicted, system 100 may include multiple computing devices that receive and transmit data associated with a prescribed treatment protocol. Examples of computing devices 120 and 121 include a laptop computer, a tablet computer, a smartphone, a desktop computer, and any other mechanism used to access networks, software, and applications. An example of software that executes on computing devices 120 and 121 includes an application configured to receive, analyze, and update data associated with prescribed treatment protocols, such as components of medical records, lab/test results, and biological data associated with patient 111. In one embodiment, computing device 121 may include a smartwatch, smart bracelet, or any other applicable wearable technology housing one or more sensors configured for contact with patient 111 further configured to receive and transmit biological data via network 106. Examples of biological data received by computing device 121 may include but is not limited to heartrate, blood pressure, body temperature, glucose level, body mass index (BMI), or any other type of measurement associated with biology. Biological data collected and transmitted by computing device 121 may be stored in database 104 and associated with one or more profiles of patient 111 utilized by healthcare professional 110.

In one embodiment, network 106 is configured to utilize components of computing devices 120 and 121 such as cameras, microphones, and any other technologies known in the art in order to establish a live communication session between healthcare professional 110 and patient 111. Healthcare professional 110 and patient 111 may further utilize network 106 to exchange text, audio files, videos, and any other applicable form of media content. Network 106 may be a packet switched network, such as the Internet, or any local area network, wide area network, enterprise private network, cellular network, phone network, mobile communications network, or any combination of the above.

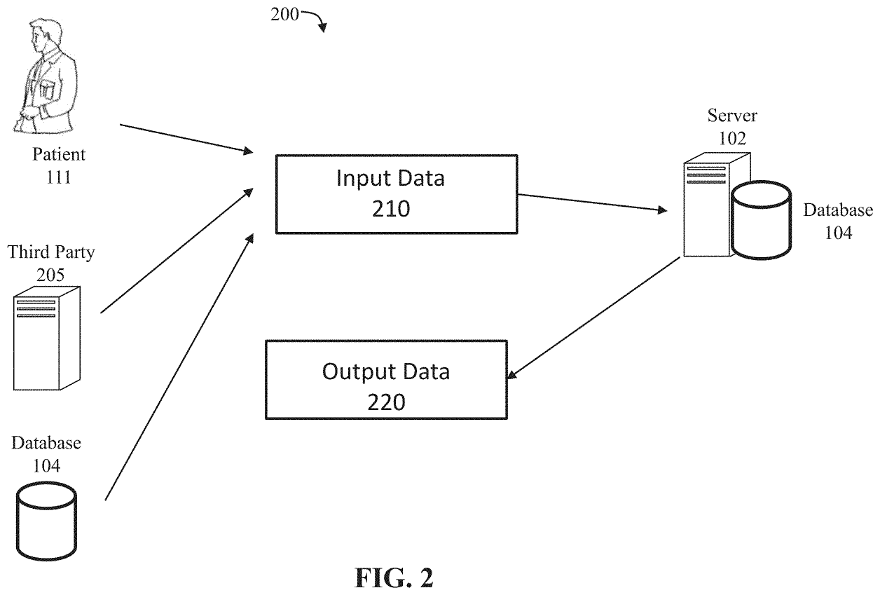

Server 102 includes a software engine that delivers applications, data, program code and other information to computing devices 120, 121. The software engine of server 102 may perform other processes such as transferring multimedia data, such as audio and video, in a stream of packets that are interpreted and rendered by a software application as the packets arrive. Server 102, and computing devices 120, 121 may each include program logic comprising computer source code, scripting language code or interpreted language code that perform various functions of the claimed subject matter. In one embodiment, the aforementioned program logic may comprise program module 507 in FIG. 2.

System 100 may be used when computing devices 120, 121, transfer data to and from database 104 coupled to server 102. Various types of data may be stored in the database 104 of server 102. For example, the database 104 may store one or more patient records for each patient, i.e., a patient record. A patient record may include personal data for the patient 111, which may include contact information for a patient 111, a medical history of the patient, demographic data of the patient, clinical data of the patient, and psychological data of the patient and occupational data of the patient. A patient record may also include assessment data for the patient, wherein the assessment data includes medical assessment data of the patient, functional assessment data of the patient, psychological assessment data of the patient and economic assessment data of the patient, or the like.

Additionally, a patient record may also include one or more prescribed regimens for a patient, which have been assigned or prescribed by a healthcare professional 110, such as a doctor or physician's assistant. A prescribed regimen includes one or more medications, as well as a definition of how often and how long the patient must take the medication. Moreover, a patient record may also include compliance data, wherein the compliance data defines the patient's level of compliance with the prescribed regimen.

In one embodiment, compliance data may be derived from data collected from computing device 121 via inputs from patient 111. Compliance data may also be derived from one or more analyses performed by network 106 with the assistance of healthcare professional 110 based on biological data retrieved by computing device 121. For example, healthcare professional 110 could initially create a prescribed treatment protocol including one or more prescribed regimens for patient 111 seeking to lower the high blood pressure of patient 111. System 100 allows computing device 121 to collect the blood pressure of patient 111 periodically or integrate other relevant extractable biological data in order for healthcare professional 110 to generate one or more analyses necessary to determine if patient 111 is in compliance with the prescribed treatment protocol. Based off of the one or more analyses, healthcare professional 110 may contact patient 111 or send an alert to computing device 121 to remind patient 111 to follow or make adjustments to the one or more prescribed regimens.

Note that although server 102 is shown as a single and independent entity, in one embodiment, the functions of server 102 may be integrated with another entity, such as the computing devices 120, 121. Further, server 102 and its functionality, according to a preferred embodiment, can be realized in a centralized fashion in one computer system or in a distributed fashion wherein different elements are spread across several interconnected computer systems.

In one embodiment, system 100 includes an application executed by healthcare professional 110. The application is configured for providing a dashboard that displays real-time data regarding patient compliance with a prescribed treatment protocol by individual and by group. The application is also configured for providing a dashboard that displays real-time data regarding sales of medication and supplements to patients with a prescribed treatment protocol by individual and by group. The application is further configured for providing a dashboard that displays real-time data regarding patient-derived revenue by individual and by group. The application may also provide a dashboard that allows the healthcare professional 110 to sort and view patients and related statistics using any of the data above. Lastly, the application may provide a communication functionality that allows the healthcare practitioner to communicate directly with their patients, such as a telecommunication session hosted by network 106 or any other carriers configured to host telecommunication services.

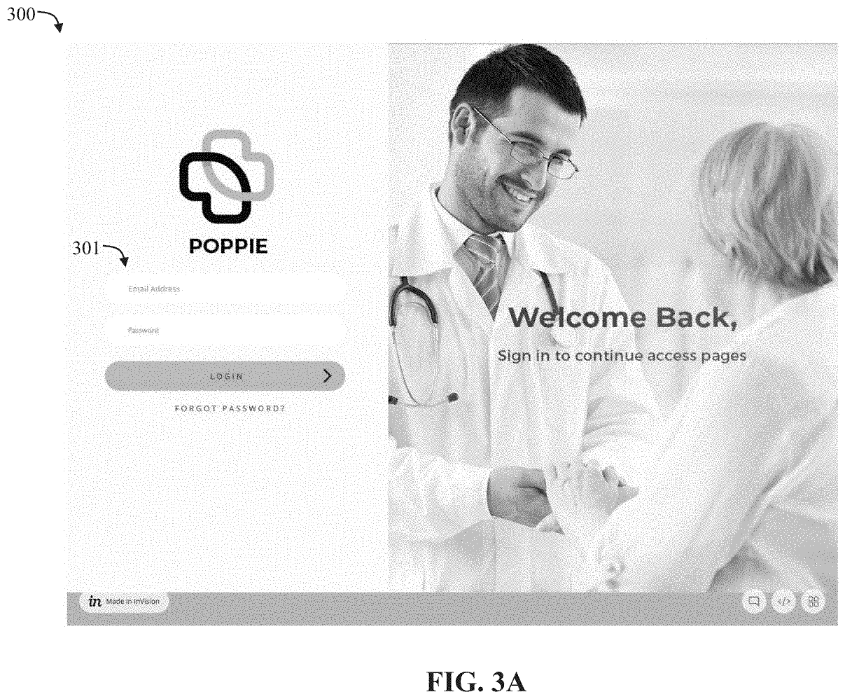

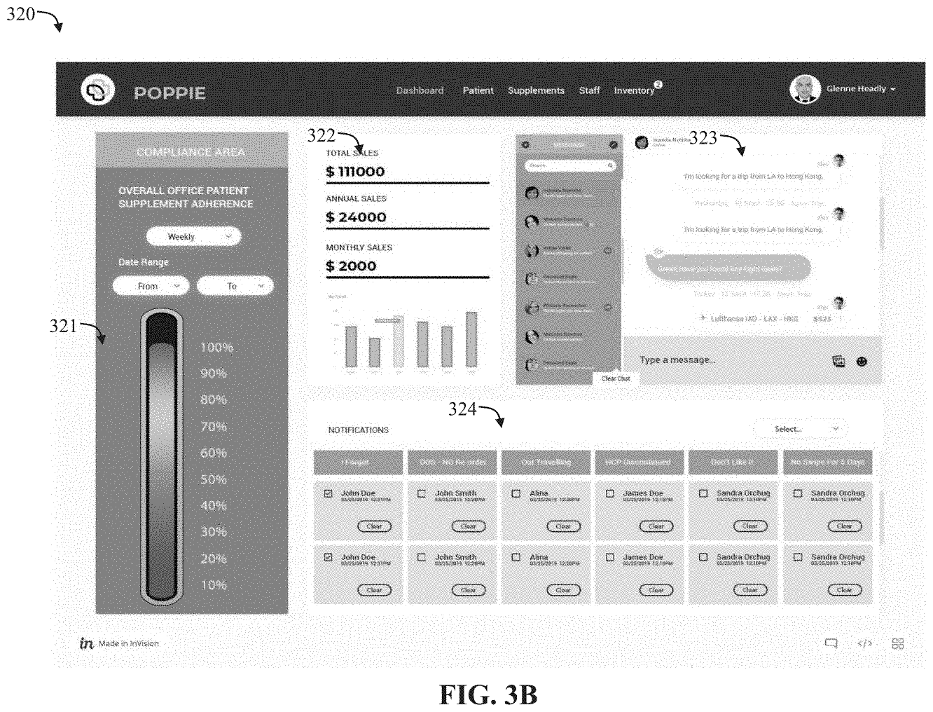

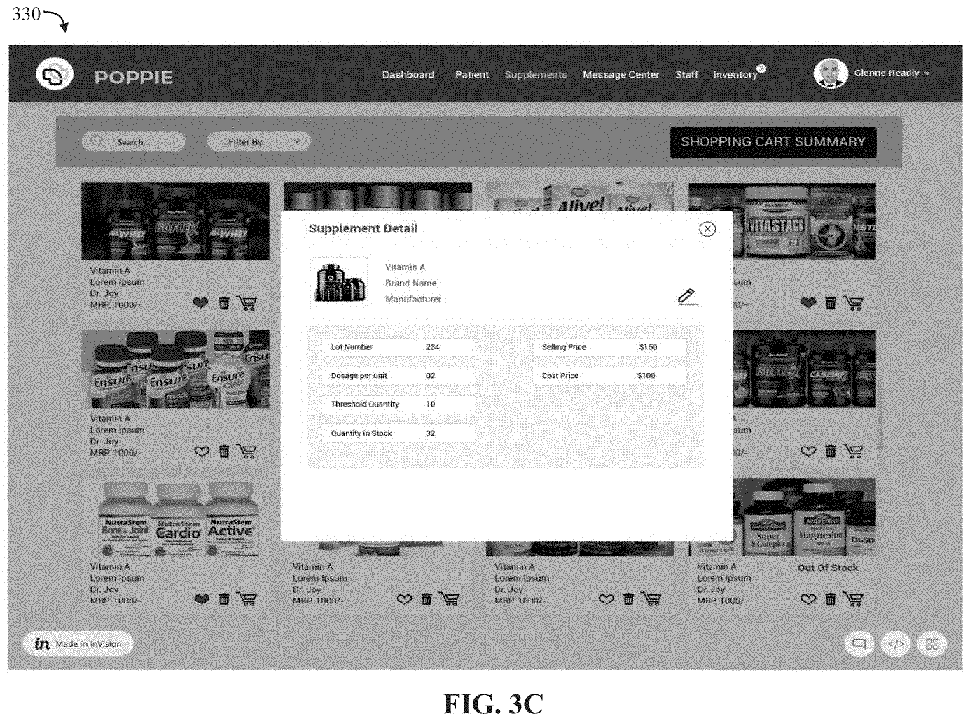

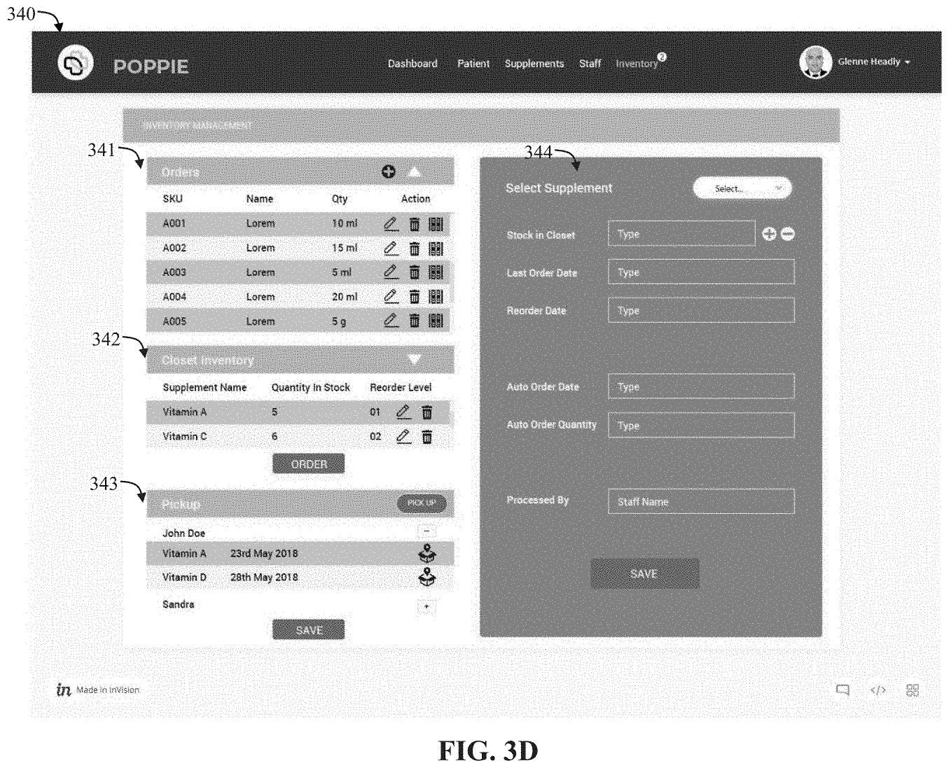

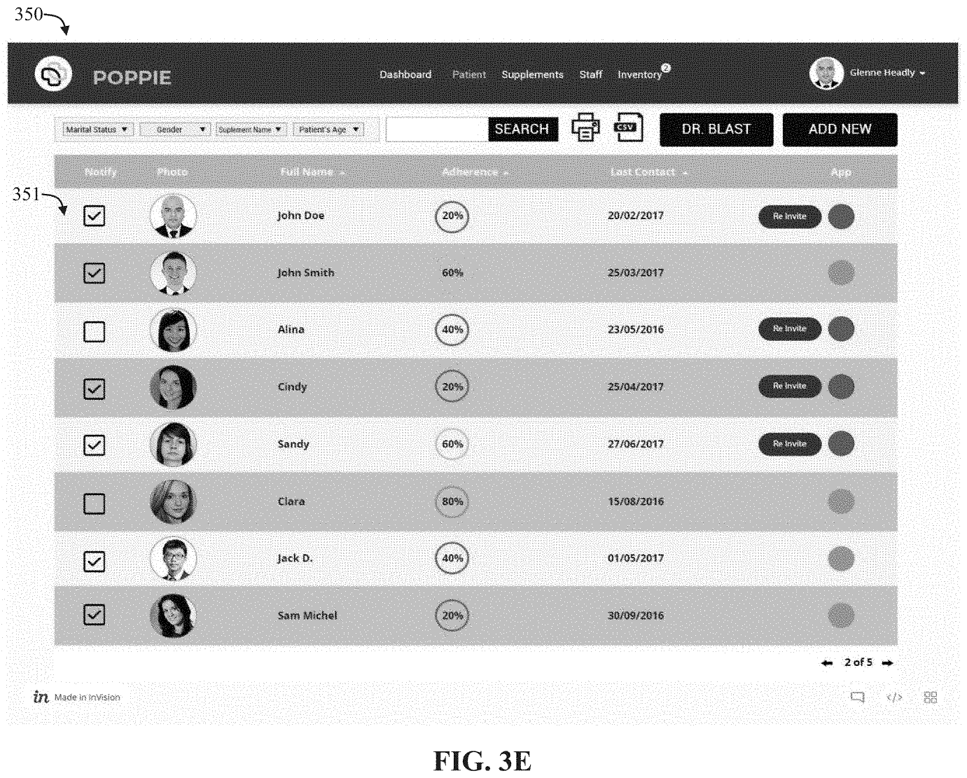

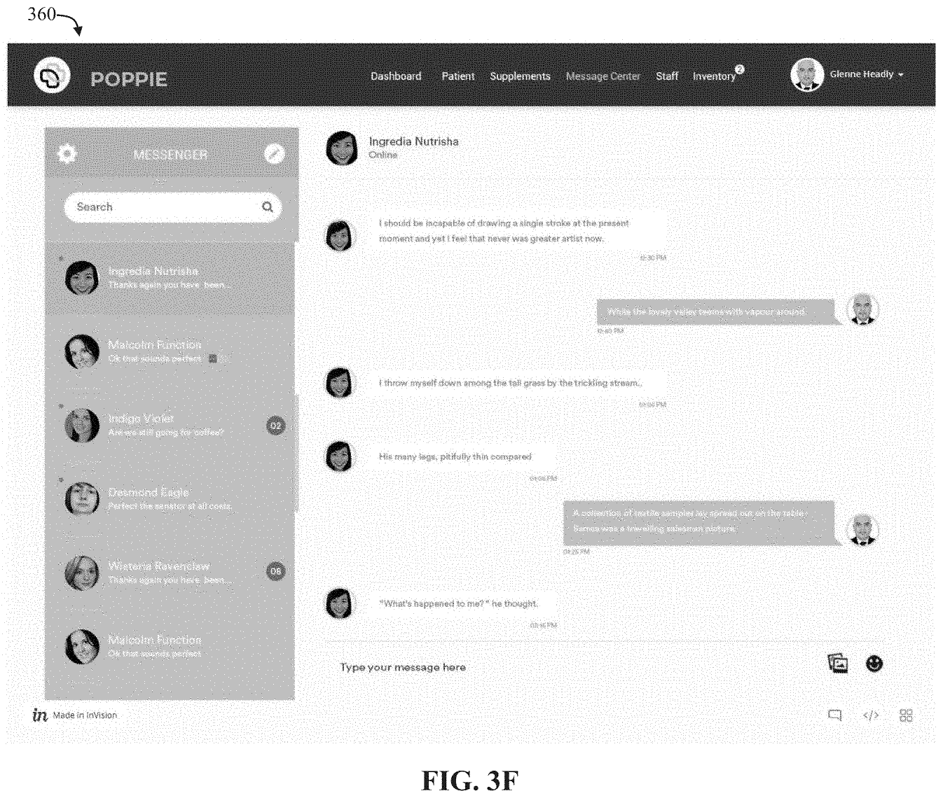

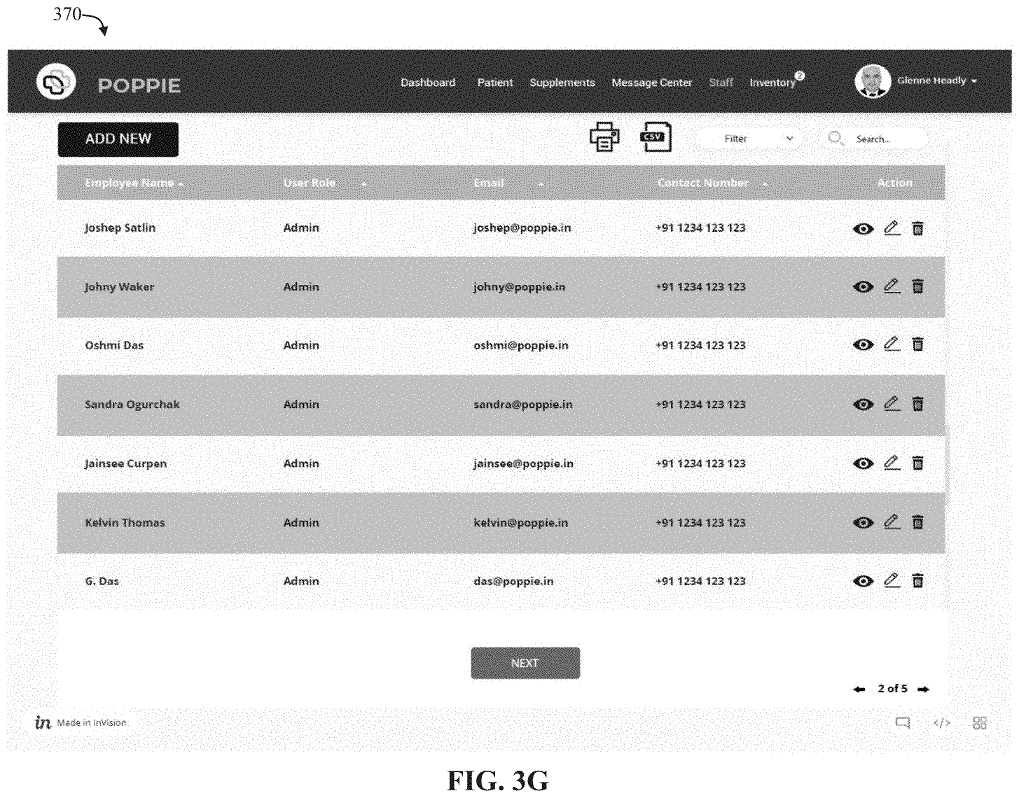

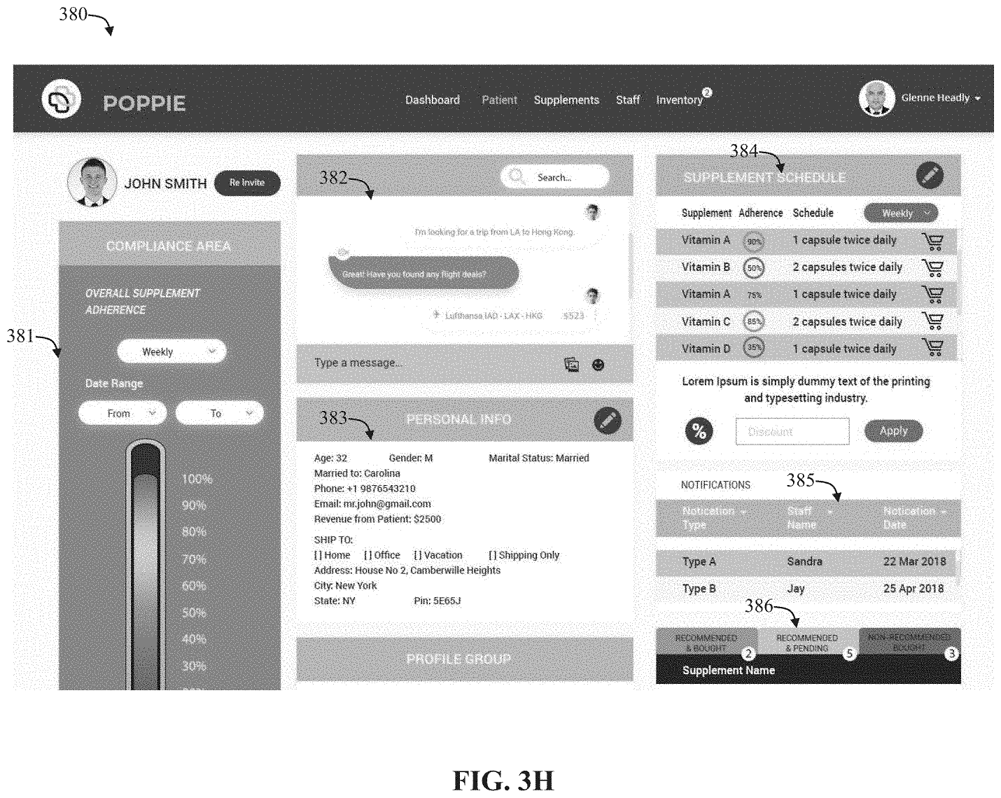

Referring now to FIG. 2, system 100 utilizes data flow 200 via including a mobile application executed by the patient 111. The mobile application may be configured for providing an interface to allow the patient to enter his compliance with a prescribed treatment protocol, and for providing an interface to allow the patient to communicate directly with their healthcare practitioner. The mobile application may also be configured for providing an interface to allow the patient to link or sync their mobile application with other health devices/trackers, and for providing an interface to allow the patient to purchase medication and supplements associated with their prescribed treatment protocol. Data flow 200 further utilizes server 102 connected to the application and the mobile application. Server 102 is configured for storing a user profile for each healthcare practitioner and each patient in database 104. In one embodiment, biological data extracted from device 121 may be integrated into the user profiles associated with patient 111. Server 102 is also configured for storing (in database 104) and serving any data related to any dashboard above. Server 102 is also configured for providing a communications platform for allowing any healthcare professional 110 to communicate directly with patients 111, said platforms include but are not limited to telecommunications, short message service (SMS), and any other applicable form of communication between two entities. Patient 111 may enter input data 210 into a first client module on computing device 121. In one embodiment, input data 210 may be supplemented from a third party source 205 configured to provide information associated with patient 111 and/or the prescribed treatment protocol. In one embodiment, input data 210 may be extracted from patient 111 or computing device 121 affixed to patient 111 and sent to server 102 via data packets or any other applicable mechanism. For example, input data 210 may be biological data of patient 111 extracted and processed from applicable wearable technology physically and communicatively coupled to patient 111 in order to assist healthcare professional 110 with determining whether patient 111 is in compliance with the prescribed treatment protocol. Input data 210 may include but is not limited to medical data, biological data, historical data, or any other applicable data associated with patient 111 and the prescribed treatment protocol. Input data 210 is transmitted to server 102 and stored in database 104. In one embodiment, server 102 may include one or more artificial intelligence engines or machine learning components in order to generate predictions, analyses, reports, alerts, analytics, or goals associated with the prescribed treatment protocol based on input data 210. Server 102 and database 104 are further configured to transmit output data 220 so that it is displayed to patient 111 on computing device 121 and/or healthcare professional 110 on device 120. In one embodiment, healthcare professional 110 may access and make amendments to data collected by server 102 before output data 220 is presented to patient 111. Output data 20 may include any or all of the data displayed in the screens shown in FIGS. 3A-3G below.

Referring now to FIGS. 3A-3G, graphical user interfaces associated with the method and system for increasing compliance with a prescribed treatment protocol are presented. FIG. 3A depicts a login page 300 of the mobile applications presented to computing devices 120 and 121. Once patient 111 or healthcare professional 110 provides access credentials to system 100 via text fields 301, a main patient dashboard is presented to computing device 121. In the case of healthcare professional 110 providing access credentials to system 100, main dashboard 320 (as illustrated in FIG. 3B) is presented to computing device 120. Main patient dashboard is configured to function as a hub in order to see both current and historical data relating to compliance with the prescribed treatment protocol. The user may further view real-time data regarding sales of medication and supplements to patients based on input data 210. For example, if the prescribed treatment protocol indicates that patient 111 has high blood-pressure, then main patient dashboard may include a plurality of offers associated with supplements configured to reduce high blood-pressure. Main dashboard 320 is configured to function as a hub for healthcare professional 110 in order to see the overall compliance rate with the prescribed treatment protocols and overall sales of medications and supplements.

Section 321 of dashboard 320 displays a digital thermometer that shows overall patient supplement compliance/adherence for all patients that are using the application over a changeable period of time (weekly, monthly, quarterly, annually, custom time frame). This data is collected through the patients' day-to-day interactions with the mobile application on the device 121 from self-reporting when they are taking a supplement. This data is important because increased adherence causes better outcomes in patient care and increases supplement sales which most functional practices sell for profit. Testing shows an increase in supplement adherence from the national average of patient adherence when patients are prompted to enter their adherence into an application. This is significant in the health care industry, because better adherence results in better health of patients. This is also significant for the health insurance industry, since health insurance plans are modifying their reimbursement to the retail pharmacies for claims based on proportion of days covered, which is a calculation based on the patient's last refill. The future may require companies and health care providers to have this personalized and detailed information about each of their patients and their individual adherence. In one embodiment, the mobile application on the device 121 of the patient provides patient notifications (to take supplements or medications) on the lock screen of the device 121. Said notifications may be configured to allow the patient to swipe or press the notification to indicate whether the patient has taken his medication or supplement. This is a quick and easy feature that saves time for the patient, as it eliminates the need for the patient to log in to his phone, or enter a password or code, which is time consuming and tedious.

In one embodiment, compliance or adherence of a patient may be calculated as followed. In a situation where a patient is not entering his compliance regularly using a mobile application (as described herein), the server 102 may make a calculation at the time the patient orders a new batch of medication or supplements. The server may calculate the grand total (GT) number of units of medication or supplements that the patient should have taken, according to the prescribed protocol, from the last date of order to the current date of order. The server may then compare the number of units of medication or supplements in the patient's possession (N) at the last date of order to the grant total that was calculated. The comparison can be used to calculate percentage of days the patient adhered to the prescribed protocol (N/GT), the total number of days the patient adhered (N) since the last date of order, the total number of days the patient did not adhere (GT-N), etc.

Section 322 of dashboard 320 displays a sales area that shows the office's total sales purchased through the application and the physical closet of inventory held in the office. Section 323 of dashboard 320 displays a messenger area that allows the practitioner to direct messages from their dashboard to patients using a large number of filters to customize the audience and allows the patients to message back from their application. Section 324 of dashboard 320 displays an alert area that serves as an alert center where the healthcare professional can take action based on real time responses and items indicated by the patient. Primarily, action occurs when a patient indicates that they didn't take a particular supplement and the patient is asked a list of questions aimed at determining why the patient didn't take the supplement. The medical office staff can take whatever action necessary to assist the patient at increasing adherence. This data is important because the national average of patient adherence to a treatment protocol is roughly 50% and it is important to know why patients aren't taking their supplements. Also this is a second layer of accountability because at any point in time the healthcare professional can take a live look at each individual patient and see where they are with their supplements, instead of waiting 3 months until their next meeting to ask them.

FIG. 3C depicts a supplement dashboard 330 configured to provide patient 111 with more detailed information regarding supplements associated with the prescribed treatment protocol. For each supplement presented, the user may view the supplement's lot number, dosage per unit, threshold quantity, quantity in stock, selling price and cost price. For each supplement presented, the user may also purchase the supplement or supplements.

FIG. 3D is an inventory dashboard 340 configured to be presented to computing device 120, which allows healthcare professional 110 to keep track of not only the stock and supply relating to medicine, supplements, or any other alimentary components, but also the rationing and distribution history of said alimentary components to patient 111. Section 341 of dashboard 340 displays the current orders for each supplement and the quantity for each order. Section 342 of dashboard 340 displays a list of the closet inventory for each supplement and the quantity in stock for each. Section 343 of dashboard 340 displays a list of supplements that have been picked up by patients, including a date, a patient name and a supplement name. Section 344 of dashboard 340 displays a list of data for each supplement, including the amount of stock in the closet, the last order date, the reorder date, the auto order date, the auto order quantity and the name of the person that processes the order.

FIG. 3E depicts a patient dashboard 350 presented to computing device 120. Patient dashboard 350 is configured to allow healthcare professional 110 to keep track of interactions with patient 111 and monitor the compliance with the prescribed treatment protocol. For each patient in the list 351 of dashboard 350 the list provides a patient name, a patient photo, a patient adherence percentage, a last contact date and a button for inviting the patient to the mobile application.

FIG. 3F depicts a message center 360 presented to computing device 120. Message center 360 is configured to allow healthcare professional 110 to initiate and monitor communications with patient 111 based on compliance or non-compliance with the prescribed treatment protocol. Communications may accomplished by short message service (SMS), multimedia messaging service (MMS), enhanced messaging service (EMS), videotelephony, or any other applicable form of communication between two entities.

The patients listed in the message center can be sorted based on adherence, marital status, gender, and age. Blast messages can be custom allocated and patients individually selected to be sent a message. This will be useful if a group of patients currently taking something needs to be notified of a change or if a particular group's adherence is particularly low or high—congratulatory or encouraging messages can be sent to said selected patients.

FIG. 3G depicts a staff listing dashboard 370 presented to computing device 120. Staff listing dashboard 370 is configured to allow healthcare professional 110 to designate and monitor access privileges of employees based upon employee allocation to the data associated with patient 111 based on the prescribed treatment protocol. For example, an employee or staff may have access to a subset of patients based on a prescribed treatment protocol shared among the subset.

FIG. 3H is a patient dashboard 380 configured to be presented to computing device 120 for viewing patient data. Section 381 of dashboard 380 displays a compliance area similar to section 321 of dashboard 320 with one exception, which is that it is specific only to the individual patient. Section 382 of dashboard 380 displays a message area for individual messages and history with said patient only, and wherein the doctor can have a running message with the patient. Section 383 of dashboard 380 displays personal information for the patient, including demographic info that the office staff has entered about said patient. Section 384 of dashboard 380 displays the supplements taken by said patient, his adherence percentage and a supplement schedule. The office staff, based on healthcare professional recommendations to the patient, creates and modifies the schedule for their supplement/drug/injection consumption, which will be linked directly to that particular patient's mobile application. This section will also show patient level adherence for each individual supplement which will allow the practitioner to address any trends.

Section 384 of dashboard 380 has capacity to indicate how said patient will interact with the application and ultimately how adherence is calculated. For example, if the patient is uploading adherence data daily, then adherence will be calculated real time. If the patient simply wants to be reminded, then adherence will be calculated based on days covered, and this will be done based on dates of purchase in the application. Even if the patient decides to walk into the office to buy the supplement, the office staff will enter said information and complete the purchase in the application so it will correctly register adherence for that particular supplement for that particular patient.

All the patient has to do is enter data to indicate they have taken a supplement. One of the largest deterrents to patients using mobile technology to keep track of adherence is they were required to know how often they were required to take the supplement and had to manually add the supplements and use the technology in a way that was far outside most patient's comfort zone. The claimed subject eliminates this requirement and allows the patient to simply enter data to indicate they have taken a supplement.

Section 385 of dashboard 380 is an alert section similar to section 324 of dashboard 320 but differs in that the alerts are only for that particular patient. Section 386 of dashboard 380 is a sales section that calculates what a particular patient is purchasing based on what was recommended, in addition to extra purchases from the store.

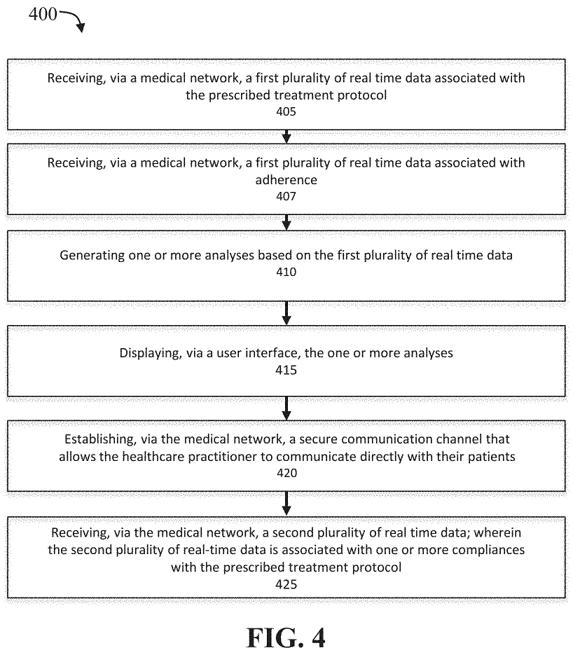

Referring now to FIG. 4, is a block diagram depicting a process 400 for increasing compliance with a prescribed treatment protocol. The hereinafter disclosed steps need not be performed in any particular order. At block 405, network 106 receives real-time data relating to a prescribed treatment protocol. In one embodiment, the prescribed treatment protocol is provided by healthcare professional 110 or third party 205, which may be, but is not limited to, a hospital network, medical records network, or any other applicable source for verified patient data. In one embodiment, the received prescribed treatment protocol may be an updated version of a previously prescribed treatment protocol. A prescribed treatment protocol may include a supplement or drug indicator, a dosage, a number of times per day or week, a total number of doses, a time cap on the regimen or protocol, and additional information, such as cautionary warnings, emergency numbers, etc.

At block 407, network 106 receives real-time data relating to adherence. In one embodiment, the real-time data is provided by patient 111. Adherence data may include a supplement or drug indicator, a dosage, a number of times taken, a total number of doses taken, a time stamp, and additional information, such as side effects, etc. All the patient has to do is enter data to indicate they have taken a supplement.

At block 410, one or more analyses associated with the prescribed treatment protocol and the adherence data are generated based on the received real-time data. In one embodiment, the machine learning components may assist healthcare professional 110 in generating the one or more analyses. At block 415, the one or more analyses are presented to computing device 121 via a graphical user interface (see data displayed in FIGS. 3A-3H). Based on the one or more analyses, healthcare professional 110 can monitor and scrutinize any data relating to the prescribed treatment protocol and the adherence data and make adjustments to the data accordingly. For example, if patient 111 is deemed at risk or subject to one or more additional ailments, based on the one or more analyses, then healthcare professional 110 may adjust or update prescribed treatment protocol and transmit the adjusted or updated prescribed treatment protocol to computing device 121 via network 106. Concurrently, supplement dashboard 330 may be updated accordingly based on the adjusted or updated prescribed treatment protocol, which allows patient 111 to view new supplements associated combating the one or more additional ailments.

At block 420, healthcare professional 110 may establish a telemedicine session with patient 111 by computing device 120 establishing a telecommunication session with computing device 121 over network 106, which allows patient 111 to ask any questions relating to the prescribed treatment protocol. In one embodiment, healthcare professional 110 may interact with patient 111 via a message portal (as illustrated in FIG. 3F) in order to update or notify patient 111 regarding any information associated with the prescribed treatment protocol. In one embodiment, healthcare professional 110 may add one or more other authorized healthcare professionals to the telecommunication session as long as the additional healthcare professionals have been added and authorized to treat the applicable individual or group via staff listing dashboard 370.

At block 425, a second set of real-time data associated with the prescribed treatment protocol is received via network 106. In one embodiment, the second set of real-time data is sourced from computing device 121 and is directly associated with whether or not patient 111 is in compliance with the prescribed treatment protocol. For example, computing device 121 may be a plurality of sensors or a wearable device worn by patient 111 configured to detect and transmit the blood pressure of patient 111. Based on the current blood pressure data, healthcare professional 110 may determine whether or not patient 111 is in compliance with the prescribed treatment protocol and assign a metric of compliance to patient 111 (as illustrated in FIG. 3E). In one embodiment, the second set of real-time data may be gathered based on a prompt or alert sent from healthcare professional 110. In one embodiment, network 106 may periodically prompt for or automatically extract (if patient 111 is wearing a biological data measuring device) the second set of real-time data based on a significant deviation from compliance with the prescribed treatment protocol. In one embodiment, if the metric of compliance reaches a predetermined threshold determined by healthcare professional 110, then main dashboard 320 and supplement dashboard 330 are updated accordingly.

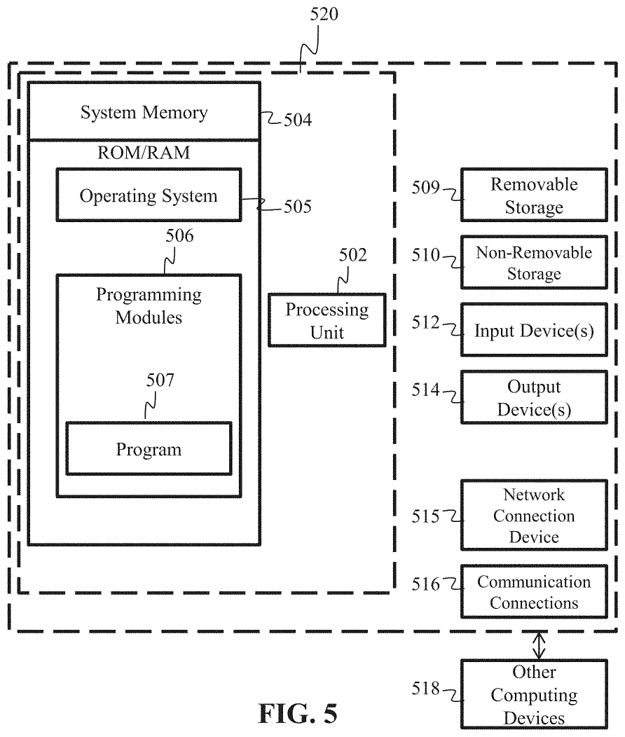

FIG. 5 is a block diagram of a system including an example computing devices in system 100 and other computing devices. Consistent with the embodiments described herein, the aforementioned actions performed by devices 102, 120, 121 may be implemented in a computing device. The aforementioned system, device, and processors are examples and other systems, devices, and processors may comprise the aforementioned computing device. The aforementioned processes may operate in other environments and are not limited to computing devices mentioned herein.

With reference to FIG. 5, a system consistent with an embodiment may include a plurality of computing devices. In a basic configuration, computing devices in system 100 may include at least one processing unit 502 and a system memory 504. Depending on the configuration and type of computing device, system memory 504 may comprise, but is not limited to, volatile (e.g. random access memory (RAM)), non-volatile (e.g. read-only memory (ROM)), flash memory, or any combination or memory. System memory 504 may include operating system 505, and one or more programming modules 506. Operating system 505, for example, may be suitable for controlling operations of computing devices in system 100. In one embodiment, programming modules 506 may include, for example, a program module 507 for executing the actions of devices 102, 120, 121. Furthermore, embodiments may be practiced in conjunction with a graphics library, other operating systems, or any other application program and is not limited to any particular application or system.

Computing devices in system 100 may have additional features or functionality. For example, computing devices in system 100 may also include additional data storage devices (removable and/or non-removable) such as, for example, magnetic disks, optical disks, or tape. Such additional storage is illustrated in FIG. 5 by a removable storage 509 and a non-removable storage 510. Computer storage media may include volatile and nonvolatile, removable and non-removable media implemented in any method or technology for storage of information, such as computer readable instructions, data structures, program modules, or other data. System memory 504, removable storage 509, and non-removable storage 510 are all computer storage media examples (i.e. memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing devices in system 100. Any such computer storage media may be part of computing devices in system 100. Computing devices in system 100 may also have input device(s) 512 such as a keyboard, a mouse, a pen, a sound input device, a camera, a touch input device, etc. Output device(s) 514 such as a display, speakers, a printer, etc. may also be included. Computing devices in system 100 may also include a vibration device capable of initiating a vibration in the device on command, such as a mechanical vibrator or a vibrating alert motor. The aforementioned devices are only examples, and other devices may be added or substituted.

Computing devices in system 100 may also contain a network connection device 515 that may allow the devices to communicate with other computing devices 518, such as over a network in a distributed computing environment, for example, an intranet or the Internet. Device 515 may be a wired or wireless network interface controller, a network interface card, a network interface device, a network adapter or a LAN adapter. Device 515 allows for a communication connection 516 for communicating with other computing devices 518. Communication connection 516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 504, including operating system 505. While executing on processing unit 502, programming modules 506 (e.g. program module 507) may perform processes including, for example, one or more of the stages of the aforementioned processes as described above. The aforementioned processes are examples, and processing unit 502 may perform other processes. Other programming modules that may be used in accordance with embodiments herein may include electronic mail and contacts applications, word processing applications, spreadsheet applications, database applications, slide presentation applications, drawing or computer-aided application programs, etc.

Generally, consistent with embodiments herein, program modules may include routines, programs, components, data structures, and other types of structures that may perform particular tasks or that may implement particular abstract data types. Moreover, embodiments herein may be practiced with other computer system configurations, including hand-held devices, multiprocessor systems, microprocessor-based or programmable consumer electronics, minicomputers, mainframe computers, and the like. Embodiments herein may also be practiced in distributed computing environments where tasks are performed by remote processing devices that are linked through a communications network. In a distributed computing environment, program modules may be located in both local and remote memory storage devices.

Furthermore, embodiments herein may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments herein may also be practiced using other technologies capable of performing logical operations such as, for example, AND, OR, and NOT, including but not limited to mechanical, optical, fluidic, and quantum technologies. In addition, embodiments herein may be practiced within a general purpose computer or in any other circuits or systems.

Embodiments herein, for example, are described above with reference to block diagrams and/or operational illustrations of methods, systems, and computer program products according to said embodiments. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments have been described, other embodiments may exist. Furthermore, although embodiments herein have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the claimed subject matter.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system for use by healthcare practitioners and patients for increasing compliance with a prescribed treatment protocol, the system comprising:
a) an application executed by a healthcare practitioner, the application configured for:
1. providing a dashboard that displays real-time data regarding patient compliance with a prescribed treatment protocol by individual and by group;
2. providing a dashboard that displays real-time data regarding sales of medication and supplements to patients with a prescribed treatment protocol by individual and by group;
3. providing a dashboard that displays real-time data regarding patient-derived revenue by individual and by group;
4. providing a dashboard that allows the healthcare practitioner to sort and view patients and related statistics using any of the data above; and
5. providing a communication functionality that allows the healthcare practitioner to communicate directly with their patients;
b) a mobile application executed by a patient, the mobile application configured for:
1. providing an interface to allow the patient to enter his compliance with a prescribed treatment protocol;
2. providing an interface to allow the patient to communicate directly with their healthcare practitioner;
3. providing an interface to allow the patient to link or sync their mobile application with other health devices/trackers; and
4. providing an interface to allow the patient to purchase medication and supplements associated with their prescribed treatment protocol;
c) a central server connected to the application and the mobile application, the central server configured for:
1. storing a user profile for each healthcare practitioner and each patient;
2. storing and serving any data related to any dashboard above; and
3. providing a communications platform for allowing any healthcare practitioner to communicate directly with patients.

2. The system of claim 1, wherein the application executed by the healthcare practitioner is an application executing on a desktop computer or a mobile computing device.

3. The system of claim 2, wherein the desktop computer or the mobile computing device is communicatively coupled with a communications network, such as the Internet.

4. The system of claim 1, wherein the application executed by the healthcare practitioner updates the dashboards it displays based on updated information received form the central server.

5. The system of claim 1, wherein the mobile application executed by the patient is a mobile application executing on a smartphone.

6. The system of claim 5, wherein the smartphone is communicatively coupled with the communications network.

7. The system of claim 1, wherein the compliance information entered into the mobile application is transmitted to the central server.

8. A method for use by healthcare practitioners and patients for increasing compliance with a prescribed treatment protocol, the system comprising:
receiving, via a medical network, a first plurality of real time data associated with the prescribed treatment protocol;
generating one or more analyses based on the first plurality of real time data;
displaying, via a user interface, the one or more analyses;
establishing, via the medical network, a secure communication channel that allows the healthcare practitioner to communicate directly with their patients; and
receiving, via the medical network, a second plurality of real time data, wherein the second plurality of real-time data is associated with one or more compliances with the prescribed treatment protocol;
wherein the method is performed by one or more computing devices.

9. The method claim 8, further comprising storing the second plurality of real time data in a centralized server communicatively coupled with the medical network.

10. The method of claim 8, wherein the one or more computing devices are communicatively coupled with the medical network via a communications network, such as the Internet.

11. The method of claim 8, wherein the second plurality of real time data includes a plurality of biological data received from a health device associated with a patient.

12. The method of claim 8, wherein the medical network comprises one or more applications configured to be executed on the one or more computing devices, wherein at least one application is a mobile application.

13. The method of claim 12, wherein the mobile application is configured to be executed by a healthcare practitioner associated with the prescribed treatment protocol.

14. The method of claim 12, wherein the mobile application is configured to be executed by a patient associated with the prescribed treatment protocol based on the one or more analyses.

15. The method of claim 12, wherein the medical network further comprises at least one server, wherein the at least one server is communicatively coupled to the one or more applications and configured for storing and updating a user profile for each healthcare practitioner and each patient.

16. The method of claim 8, wherein the secure communication channel is displayed on one or more user interfaces associated with the one or more computing devices.

17. The method of claim 8, wherein at least one of the computing devices is configured to provide the second plurality of real time data to the medical network.

18. A system for use by healthcare practitioners and patients for increasing compliance with a prescribed treatment protocol, the system comprising:
at least one server designed and configured to:
receive a first plurality of real time data associated with the prescribed treatment protocol;
generate one or more analyses based on the first plurality of real time data;
display the one or more analyses;
establish a secure communication channel that allows the healthcare practitioner to communicate directly with their patients; and
receive a second plurality of real time data, wherein the second plurality of real-time data is associated with one or more compliances with the prescribed treatment protocol.

19. The system of claim 18, further comprising a medical network communicatively coupled to the at least one server and one or more computing devices via a communications network. wherein the one or more computing devices are configured to be associated with a healthcare practitioner and a patient.

20. The system of claim 18, wherein the at least one server is configured to store and update one or more profiles associated with healthcare practitioners and patients based on the one or more compliances with the prescribed treatment protocol.
